(19) United States
(12) Patent Application Publication
(10) Pub. No.: US 2017/0014018 A1
(43) Pub. Date: Jan. 19, 2017

(54) APPARATUS AND METHOD FOR ALIGNING STEREOSCOPIC LENS ASSEMBLIES

(71) Applicant: TransEnterix Surgical, Inc., Morrisville, NC (US)

(72) Inventor: Brian P Cash, Cary, NC (US)

(21) Appl. No.: 15/212,150

(22) Filed: Jul. 15, 2016

Related U.S. Application Data
(60) Provisional application No. 62/192,689, filed on Jul. 15, 2015.

(51) Int. Cl. A61B 1/00; A61B 1/05; A61B 1/313

(52) U.S. Cl. CPC A61B 1/00096; A61B 1/313; A61B 1/00193; A61B 1/05; A61B 1/00045

(57) ABSTRACT

An adjustable assembly for a lens system comprises a lens assembly mount having a first bore and a second bore, a lens assembly that has a fixed longitudinal axis within the first bore, and an adjustable lens tube housing a second lens assembly within the second bore. The adjustable lens tube has a proximal portion, a distal portion forming a spherical joint with a distal portion of the second bore, and a longitudinal axis. A double eccentric mechanism in the second bore is in contact with the proximal portion of the lens tube, and is adjustable to pivot the lens tube within the second bore to alter the orientation of its longitudinal axis.

### BACKGROUND

Stereoscopic imaging devices typically include a pair of adjacent channels, each of which houses a separate lens assembly. Each such lens assembly forms an image path through which light passes before impinging on a corresponding image sensor such as a CMOS sensor. The relative alignment of lenses comprising the two image paths is of high importance. Even small amounts of misalignment may manifest as mild to moderate discomfort to the user of a stereoscopic imaging system. Although it is desirable for such devices to have integrated alignment features, miniature lens assemblies, such as those present in stereo endoscopes, have minimal space for dedicated alignment features, and so lens alignment is often carried out using separate alignment fixtures, electronic or software-based image alignment/adjustment, or screw-based lens positioning systems.

The invention disclosed herein provides a compact, integrated mechanism for aligning and mechanically fixing lens assemblies relative to one another for the purpose of accommodating tolerances in the manufacture and assembly of said assemblies that may cause the lens stacks to point in different directions and to align said assemblies so that they have a desired relative orientation, either parallel or otherwise.

### BRIEF DESCRIPTION OF THE DRAWINGS

FIG. 1A is a cross-section view of a lens assembly mount for a stereoscopic scope, showing the channels and the double eccentric mechanism within one of the channels.

FIG. 1B is similar to FIG. 1A, but the spherical joint and the double eccentric mechanism are not shown.

FIG. 2 is a plan view of the proximal end of the lens assembly mount shown in FIG. 1A.

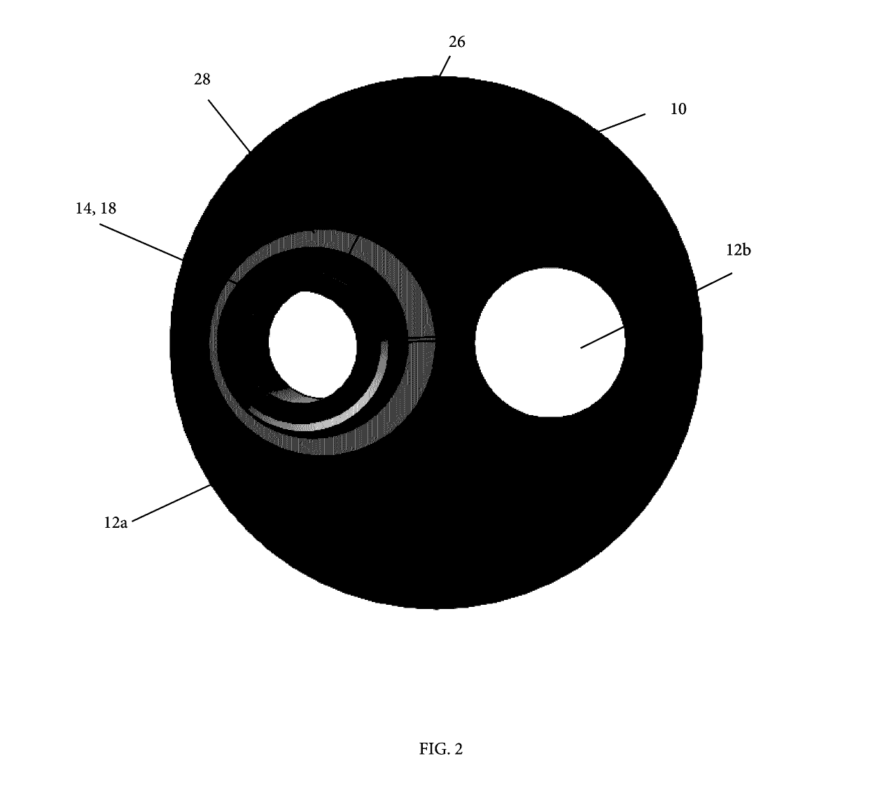

FIG. 3 is a perspective view showing the double eccentric mechanism shown in FIGS. 1A and 2.

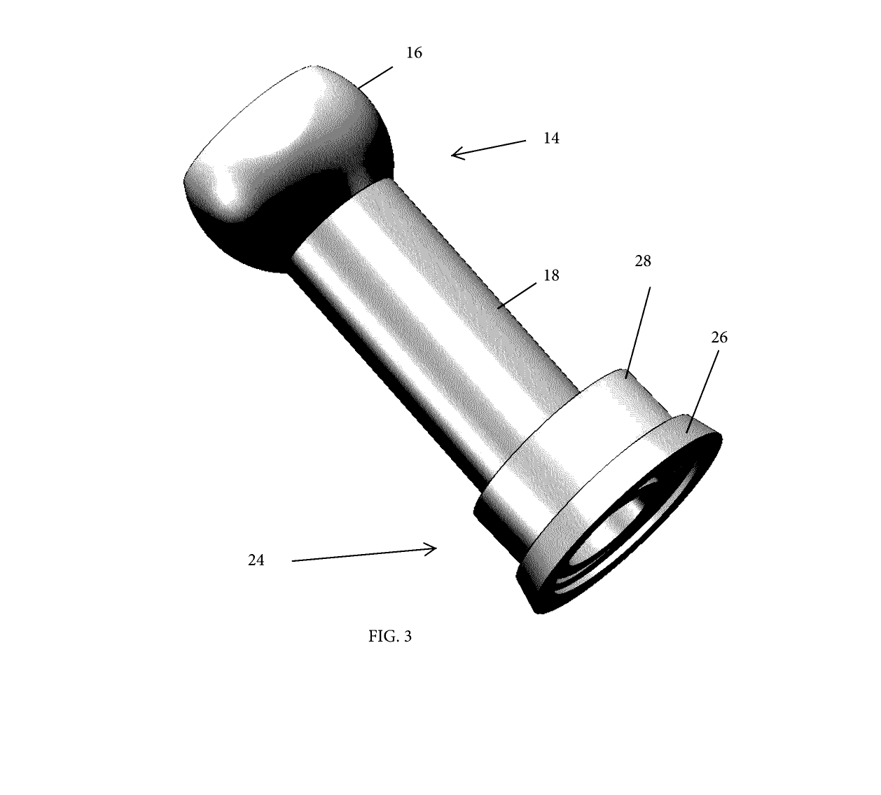

FIG. 4 is a cross-section view of the channel housing the double eccentric mechanism, and further shows an example of a mechanism for fixing the double eccentric mechanism in a selected position.

### DETAILED DESCRIPTION

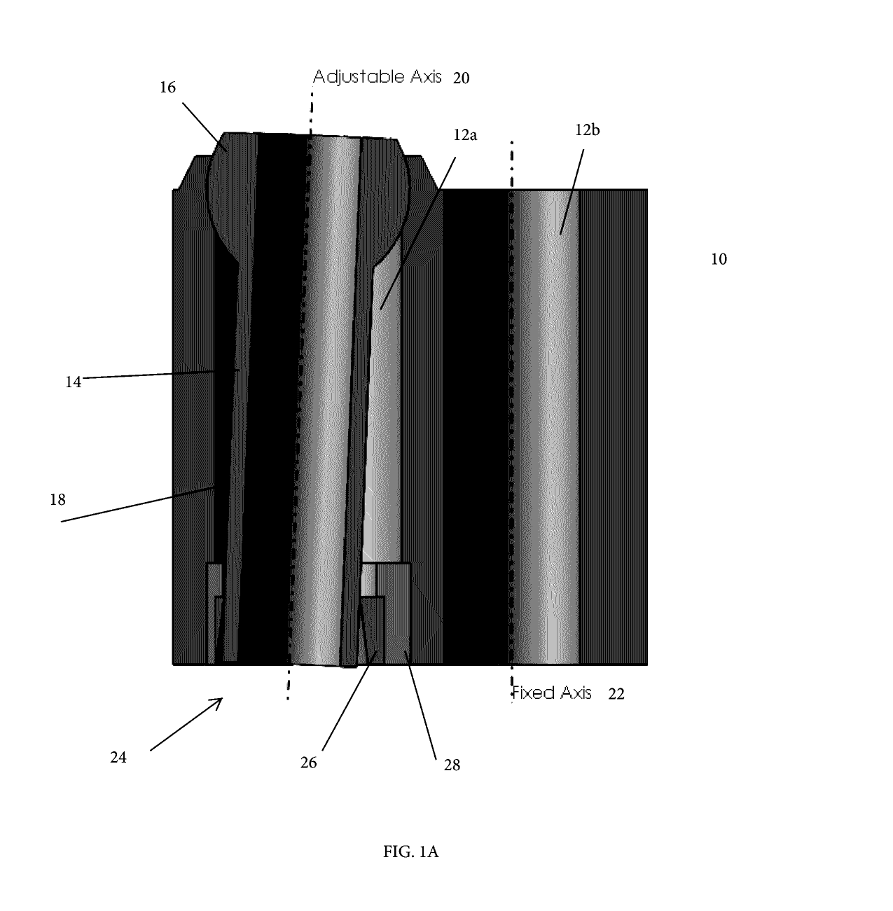

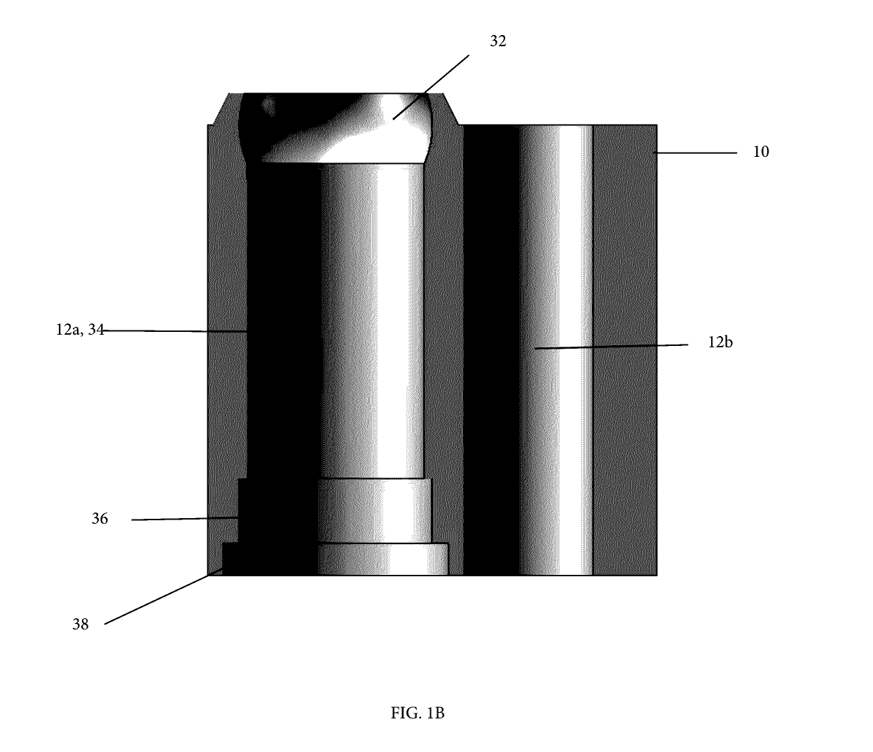

FIG. 1A shows, in cross-section view, a lens assembly mount 10 for a stereoscopic scope, such as a laparoscope or endoscope. Shaft 10 includes a pair of channels 12a, 12b. In some embodiments these longitudinal axes may be parallel to one another, while in other embodiments they might be non-parallel. As shown in FIG. 1B, channel 12a has a distal section 32 in which the channel wall has a spherical contour, and proximal sections 34, 36, 38 preferably having cylindrical walls. Those sections include a main section 34 having a diameter smaller than the diameter of the distal section 32, an increased diameter section 36 proximal to the main section 34, and a proximalmost section 38 having a larger diameter than the section 36.

Within one of the channels 12a is a tubular lens tube/element 14 having a spherical distal end 16 and a shaft section 18 that may be cylindrical. Distal end is 16 is disposed within the distal section 32 of the channel 12a, while shaft section 18 extends to the proximal section 38 as shown in FIG. 1A. Lens assembly mount 10 is designed such that a first lens assembly (or “lens stack”) is positioned in channel 12b either directly or within a stationary lens tube that is inserted into channel 12b in a fixed position so that it shares a longitudinal axis with the channel 12b. A second lens assembly is positioned in the lumen of lens tube 14. Following assembly of the lenses into the lens assembly mount 10, the lens assembly mount is positioned on a distal end of an imaging device, with the image sensor(s) proximal to the lens assemblies, in a manner known to those skilled in the art.

As discussed in the background section, manufacturing tolerances may cause the two lens assemblies to be misaligned from one another in a way that leads to discomfort to users observing the image output on a display. To allow for correction of any such misalignment, the orientation of longitudinal axis 20 of shaft section 18 is adjustable relative to the orientation of the axis of channel 12a (and thus also relative to the orientation of fixed axis 22 of channel 12b). In particular, spherical end 16 of lens element 14 forms a spherical joint within the distal section 32 of channel 12a, so that the more proximal shaft section 18 of the lens element 14 can pivot relative to the channel 12a. A double-eccentric mechanism 24 is positioned to allow the user to angularly adjust axis 20 in about three axes converging at a single point at the center of the aforesaid spherical joint. The double eccentric mechanism comprises shaft section 18, which is to be translated, and two rotational rings 26, 28 which surround and support the shaft within the section 36. Each ring has an inner circular bore that is laterally offset from the ring's rotational axis. The outer ring 28 has outer edges in contact with the walls of section 36, and it supports the inner ring 26 within its opening. The inner ring 26 supports the shaft section 18 of lens tube 14 within its own opening.

The inner ring may have an inner bore that tapers towards one of its ends to allow for minimal contact with the lens tube as shown, so as to facilitate precise edge/point contact with the lens tube and to minimize friction on the assembly. As an alternative, a second spherical joint might be positioned to slide axially on the lens tube to provide an appropriate support for the lens tube while not over constraining the assembly. As yet another alternative, a thin ring may be used.

By rotating the rings 26, 28 with respect to one another, the shaft section 18's center can be translated in two dimensions by exerting leverage on the proximal end of the shaft section 18 in order to move the lens element about the spherical joint and thus change the orientation of the lens element axis in three dimensions.

Note that the lens tube 14 may be replaced with two sliding tubes that may be used adjust the focus of the lens assembly during the alignment.

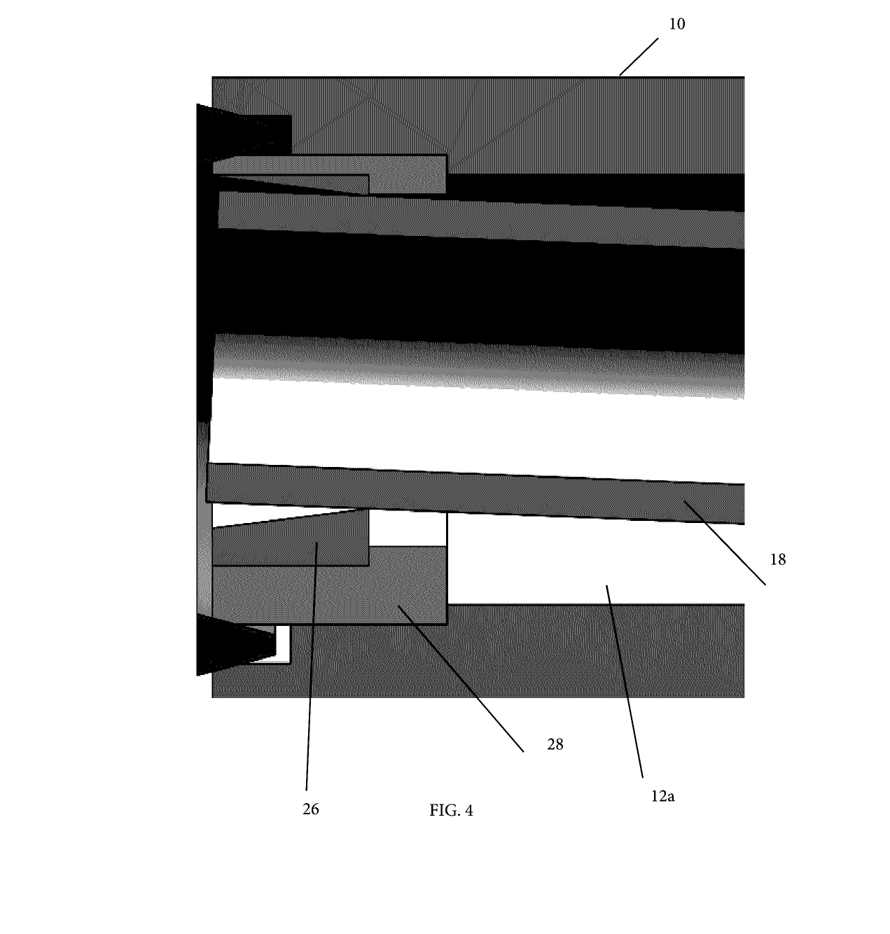

Referring to FIG. 4, a feature is provided for locking the orientation of the lens element 14. While the feature may take a variety of forms, in this embodiment the feature comprises a tapered sleeve 30 that is pressed in axially at the section 38 of the lens assembly mount 10 to frictionally engage the interior wall of section 38 and the outer edge of the ring 28, and to compress the moving elements (rings 26, 28), to prevent them from rotating, and thus to prevent unintended shifting of the axis 20 (FIG. 1A).

Various alternative locking mechanisms may be used for this purpose including, without limitation, injection of adhesives through ports or holes in the lens assembly mount, or inserting one or more threaded fasteners perpendicular to the lens assembly mount bore over the inner and outer rings such that when they are tightened, they compress the rings against one another to lock their motion.

During assembly, the double eccentric mechanism is used to move the axis 20 such that the axes 20, 22 have a desired relative alignment (whether that alignment is parallel or non-parallel). To move the axis 20, the user selectively rotates the outer ring 28 within the channel 12a of the lens assembly mount 10, and selectively rotates the inner ring 26 within the eccentric bore in the outer ring 28. By rotating the inner and outer rings with respect to the lens assembly mount and one another, the position of the inner bore of the inner ring 26 (and thus, the proximal end of the lens tube 14) can be moved in 2 dimensions, changing the angle of the adjustable axis 20 which passes through the center of the spherical joint.

Once the lens tube 14 is in the desired orientation, the double eccentric mechanism may be locked out against further rotation of the rings 26, 28 using mechanisms such as those described above. For example, the tapered ring (FIG. 4) may be pressed into the section 38 of channel 12a so as to compress the inner and outer rings to the point that the double eccentric mechanism can no longer be adjusted.

We claim:

1. An adjustable assembly for a lens system, comprising:
a lens assembly mount having a first bore and a second bore, each bore for receiving a lens assembly, the first bore having a first longitudinal axis;
an adjustable lens tube within the second bore, the adjustable lens tube having a second longitudinal axis, said lens tube having a proximal portion, and a distal portion forming a spherical joint with a distal portion of the second bore; and
a double eccentric mechanism in the second bore in contact with the proximal portion of the lens tube.

2. The assembly of claim 1, wherein the double eccentric mechanism comprises:
a circular inner ring having an outer circumference and a circular bore, wherein the center point of the circumference is laterally offset from the longitudinal axis of the circular bore, the inner ring positioned such that the proximal portion of the lens tube is disposed within the bore of the inner ring; and
a circular outer ring having an outer circumference and a circular bore, wherein the center point of the circumference is laterally offset from the longitudinal axis of the circular bore, the outer ring positioned such that the inner ring is disposed within the bore of the outer ring;
wherein rotation of the inner or outer ring pushes the proximal portion of the lens tube in a lateral direction, causing pivotal movement of the lens tube relative to the spherical joint and thereby changing the orientation of the second longitudinal axis relative to the orientation of the first longitudinal axis.

3. The assembly of claim 2, wherein in the bore of the inner ring is tapered such that only a distal or proximal edge of the bore contacts the lens tube.

4. The assembly of claim 2, further including a locking element positionable to prevent rotation of the inner and outer rings.

5. The assembly of claim 4, wherein the locking element is a tapered ring insertable into a distal end of the lens tube.

6. The assembly of claim 4, wherein the locking element is an adhesive introduced into the lens assembly mount.

7. The assembly of claim 4, wherein the locking element comprises a threaded fastener extendable through the wall of the lens assembly mount into contact with the outer surface of the outer ring.

8. A method of altering the orientation of a longitudinal axis of a lens assembly, comprising the steps of:
positioning a lens tube within a bore of a lens assembly mount, said lens tube having a proximal portion, and a distal portion forming a spherical joint with a distal portion of the bore, said lens tube further having a longitudinal axis; and
positioning a double eccentric mechanism in the second bore in contact with the proximal portion of the lens tube; and
adjusting the double eccentric mechanism to alter the orientation of the longitudinal axis.

9. The method of claim 8, wherein:
positioning the double eccentric mechanism comprises
positioning a circular inner ring such that the proximal portion of the lens tube is disposed within a circular bore of the inner ring, said inner ring having an outer circumference, wherein the center point of the circumference is laterally offset from the longitudinal axis of the circular bore; and
positioning a circular outer ring such that the inner ring is disposed within the circular bore of the outer ring, said outer ring having an outer circumference, wherein the center point of the circumference is laterally offset from the longitudinal axis of the circular bore; and
wherein the method further comprises rotating of the inner or outer ring to thereby push the proximal portion of the lens tube in a lateral direction, causing pivotal movement of the lens tube relative to the spherical joint and thereby altering the orientation of the longitudinal axis of the lens tube.

10. The method of claim 8, further comprise:
locking the position of the double eccentric mechanism so as to retain the orientation of the longitudinal axis in the altered position.
